(19) United States
(12) Patent Application Publication
(10) Pub. No.: US 2015/0196241 A1
(43) Pub. Date: Jul. 16, 2015

(54) Startscreen Diagnostics

(71) Applicant: Ziv Yekutieli, Zichron Yaakov (IL)

(72) Inventor: Ziv Yekutieli, Zichron Yaakov (IL)

(21) Appl. No.: 14/578,364

(22) Filed: Dec. 20, 2014

(30) Foreign Application Priority Data
Jan. 14, 2014 (US) 61920036

(51) Int. Cl. A61B 5/16; A61B 5/00; A61B 5/11

(52) U.S. Cl. CPC A61B 5/16; A61B 5/1124; A61B 5/4005; A61B 5/7275

(57) ABSTRACT

The sensors of a smartphone, tablet, laptop or other device is used to record user actions during attempts to unlock the device. The touch-sensitive surface of a smartphone or other touchscreen, accelerometer, magnetometer, inclinometer, gyro, GPS, camera, and microphone may amongst other sensors be used to record user responses to a login challenge. This challenge may consist of a requirement to give a correct password or numerical code; a puzzle (such as a methamatical or textual riddle), identification of digits, objects, or faces; and other challenges or actions.

### CLAIM OF BENEFIT

This application claims benefit of provisional application 61/920,036 filed 16 Jan. 2014.

### FIELD OF THE INVENTION

The present invention relates to the field of diagnostic tests, and in particular to diagnostic tests that may be carried out by means of interaction with standard smartphones, laptop computers, and desktop computers.

### BACKGROUND OF THE INVENTION

Deterioration of fine motor control, cognitive abilities, hearing, sight, and the other senses are an unfortunate fact of the aging process for many people. Such deterioration can also occur after stroke, accident, or as a result of various disease processes. The ability to track such degeneration and also to track the response of stroke victims, accident victims and the like to various treatments can serve as a vital tool for assessing the efficacy of these treatments.

Standard tests of motor control, cognitive function, and sensory acuity often require specific equipment not commonly available to the layperson, and the tests are in any case generally administered by skilled operators in clinical settings such as doctor's offices, health clinics, and the like.

Therefore it fulfills a long-felt need to allow for tests of fine motor control, sensory acuity, and cognitive function in a seamless, transparent, and effortless manner not requiring bespoke equipment nor skilled operator nor clinical setting.

### SUMMARY OF THE INVENTION

In one embodiment of the invention, the sensors of a smartphone, tablet, laptop or other device is used to record user actions during attempts to unlock the device. The touch-sensitive surface of a smartphone or other touchscreen, accelerometer, magnetometer, inclinometer, gyro, GPS, camera, and microphone may amongst other sensors be used to record user responses to a login challenge. This challenge may consist of a requirement to give a correct password or numerical code; a puzzle (such as a methamatical or textual riddle), identification of digits, objects, or faces; specific movement paths of a user's finger or fingers; specific movement paths of the device itself in space; the speaking or generation of sounds such as words or tones; the performance of specific gestures; and others including combinations of these. For example, a figure or set of points may be presented that the user must trace in order to unlock a locked device. The actual path of the user's finger or fingers as he traces through the required points (or along a curve of the required figure, as the case may be) is recorded, possibly including time information.

The user's actions are recorded to a degree of resolution that diagnostic information can be gleaned therefrom. For instance in the example above, the deviation of the user's actual path from the a given required path may be recorded as well as other statistics such as repeatability between subsequent attempts, shaking or vibration of the device, and the like.

The foregoing embodiments of the invention have been described and illustrated in conjunction with systems and methods thereof, which are meant to be merely illustrative, and not limiting. Furthermore just as every particular reference may embody particular methods/systems, yet not require such, ultimately such teaching is meant for all expressions notwithstanding the use of particular embodiments.

### BRIEF DESCRIPTION OF THE DRAWINGS

Embodiments and features of the present invention are described herein in conjunction with the following drawings:

FIG. 1 shows one possible embodiment of a tracing setup of the invention;

FIG. 2 shows a tracing setup of the invention with a traced route overlayed upon it;

FIG. 3 shows a tracing setup of the invention with several traced routes overlayed upon it.

FIG. 4 shows a possible smartphone login challenge consisting of a maze, the solution to which the user traces on the smartphone touchscreen.

FIG. 5 shows a possible smartphone login challenge consisting of a face matching task.

In the figures and/or description herein, the following reference numerals have been utilized throughout the figures:

It should be understood that the drawings are not necessarily drawn to scale.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

The invention provides a method for diagnostic evaluation of mental, motor, and sensory function in people at relatively high measurement rates of several time per day, over long periods of time (e.g. for years) in an unobtrusive manner. The general case involves the use of a login challenge to glean diagnostic information concerning a user's sensory, motor, and cognitive abilities.

For instance, a swipe path may be required. This requires recall of the swipe path, and fine motor control for the reproduction thereof. The amount of mental acuity required for this task can be made arbitrarily high; for instance the user may be required to reproduce paths generated on the fly of a given length, or may be requried to solve spatial, geometric, mathematical, or other puzzles such as those appearing in intelligence tests of various sorts.

In addition to memory and puzzle-solving tasks, any other aspect of mental operation that may be accessed by means of standard smartphone hardware falls within purview of the invention. Thus inductive reasoning, situational judgement, intelligence quotient, mental age, analogic reasoning, cognitive development, mental chronometry, and neuropsychological testing may be carried out using the invention. Elements from standard tests may be incorporated, including but not limited to standard IQ tests, Kohls block, the Miller Analogies test, Otis-Lennon, Raven's rogressive Matrices, Stanford-Binet IQ, the Wechsler Adult Intelligence Scale (and that for children and preschool), the Wonderlic test, Cambridge Neuropsychological Test, Cognitive Process Profile, Draw-a-Person Test, Knox cubes, Modern Language Aptitude, Pimsleur Language Aptitude test, Sally-Anne test, and metacognition may all contain elements suitable for use with the inventive method.

The challenge may consist for example of a requirement to give a correct password or numerical code; a puzzle (such as a methamatical or textual riddle), identification of digits, objects, or faces; answering a series of questions based on information presented, and tests of situational awareness.

Sensory acuity may also be tested, for instance by incorporating sight, sound, vibration and the like into the login challenge. Generally speaking, any of the phone sensors may be used to assess user motor abilities. For one example, hearing may be evaluated by having the device emit a series of tones at a given volume, and requiring the user to reproduce these tones by humming them, identifying them on a keyboard or the like. Vibration of the phone itself may be assessed to diagnose tremors in the hands or bodies of users. The user may be required to move the phone in a certain pattern in space with a given speed, this requiring not only fine motor control but speed to an arbitrary extent. Speed, acceleration, orientation sensitivity, gait and other aspects of a user's physiological condition may also be assessed by means of the accelerometer, orientation sensor, GPS, and other sensors that are at this point standard in smartphones and tablets. Thus for instance the user may be required to jump in place, or run a certain distance at a certain speed in oder to unlock the phone; jumping ability and gait can be measured by means of the accelerometer and magnetometer, while speed can be assessed using the GPS.

As examples, the login challenge of the inventive method may require the speaking or generation of sounds such as words or tones; the performance of specific gestures, and the like. A figure or set of points may be presented that the user must trace in order to unlock a locked device. The actual path of the user's finger or fingers as he traces through the required points (or along a curve of the required figure, as the case may be) is recorded, possibly including time information.

The user's actions are recorded to a degree of resolution that diagnostic information can be gleaned therefore. For instance in the example above, the deviation of the user's actual path from a given required path may be recorded, as well as other statistics such as repeatability between subsequent attempts, shaking or vibration of the device, and the like. The invention uses the touch-sensitive surface of a smartphone to record movement paths of a user's finger or fingers. A figure or set of points may be presented that the user must trace, for instance in order to unlock a locked device.

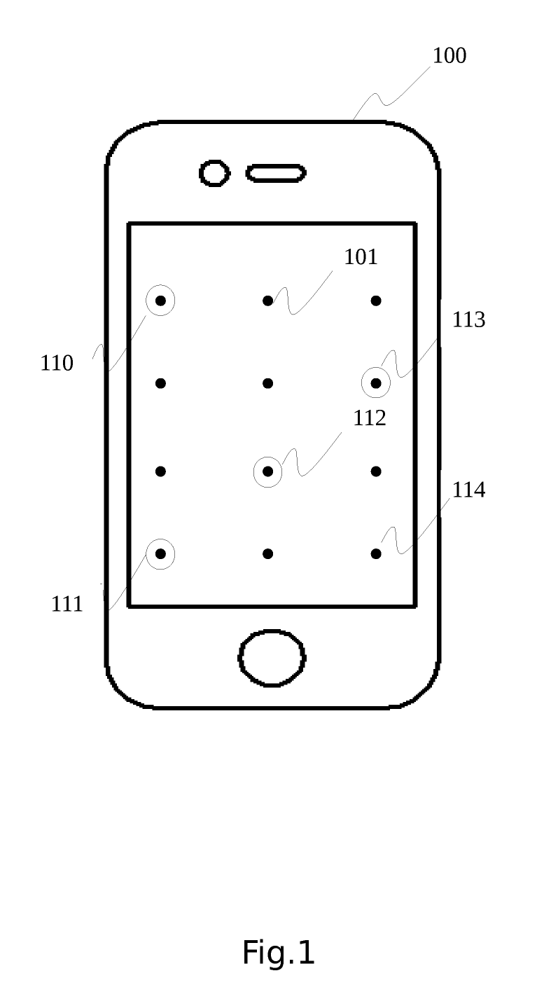

A possible setup for this task is shown in FIG. 1. Here a smartphone 100 is used to display a set of points 101 on the screen. Some ordered subset of these 110,111,112,113.114 are chosen, either by an app associated with the inventive system, or by the user. The user is tasked with tracing a continuous path through these points, in order, without lifting finger from screen.

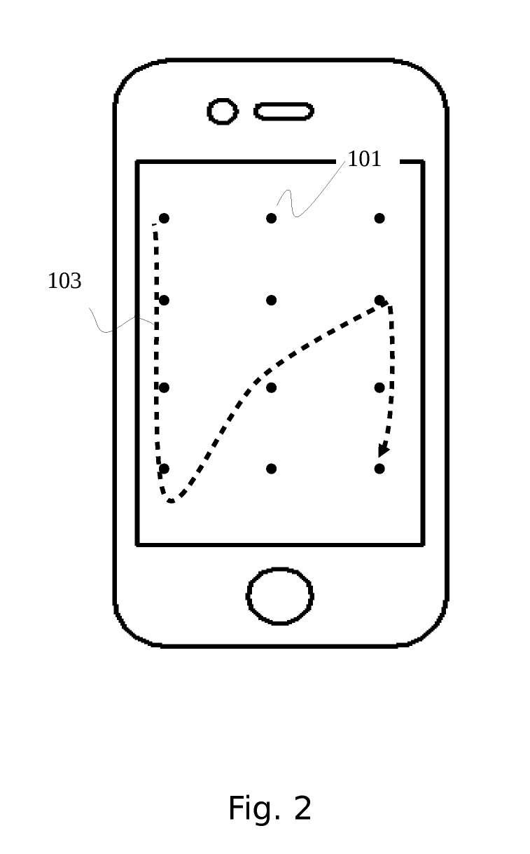

The actual path of the user's finger or fingers as he traces a path through the required points (or along a curve of the required figure, as the case may be) is recorded, possibly including time information. Such a path is shown for example in FIG. 2 as element 103. The information may be recorded as a set of triples for instance, including horizontal, vertical, and time components in the form of a vector (x,y,t). Alternatively the path may be recorded as a set of splines, with times associated with each spline, or as splines through the points in 3 dimensions (x,y,t) instead of splines through the points (x,y).

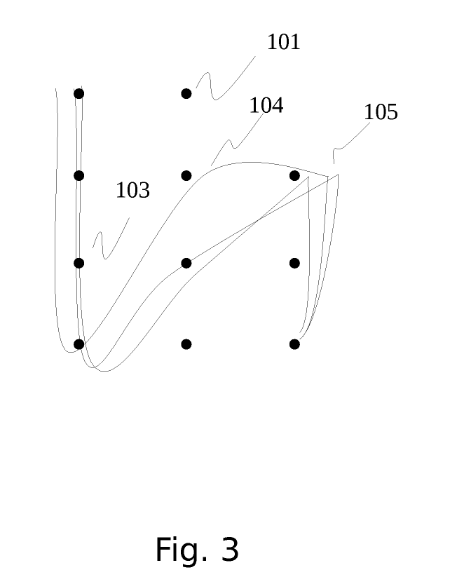

Once a path is recorded, it is stored (for example on a remote server in communication with an app of the invention running on the smartphone.) This stored pattern may be compared with previous recordings, as shown in FIG. 3; the paths 103, 105 run through the chosen points but the path 104 has a mistake. These mistakes may be recorded by the system, which optionally may be used a device unlock task. In this case, if the user makes a mistake, the device remains locked, the pass serving as a sort of entry code for unlocking.

The degree of smoothness of the curve may be recorded, as one example of a number of statistics concerning the path that may be gathered beyond whether all the required points have been hit in order. For example the curve may be fit using a best fit spline or set of splines. For a given set of fitting splines, the actual curve will deviate from the best fit by some amount. The amount of deviation may be calculated for instance by use of a mean distance measure.

As another example of a statistic that may be calculated from the curve, the total distance of the curve may be calculated, since a straighter curve will have a shorter total distance than a shaky line (drawn for example by a sufferer from Parkinson's disease).

A further statistic may be the ‘repeatability’ of a given user, or how closely they match their previous paths. This may be calculated for instance by means of a comparison between a given path and an ‘average’ path, or between a given path and all other previous paths.

As mentioned the reference path may be an average of all paths recorded, or an optimal straightest path through the desired points, or a path through the desired points having a minimal spline degree, or the like.

The calculations above may also be done in alternative fashion, for example by removing reference to time and simply comparing closest points instead of points corresponding in time. Alternatively the points may be considered as points in three dimensions (x,y,t), with three dimensional paths being compared in various ways.

As mentioned, the set of points may either be chosen by the user or automatically generated by the system. The required points may also be generated automatically and indicated only briefly, thus requiring both dexterity and memory of the user. The required points as mentioned may also lie upon a figure, for example a line drawing.

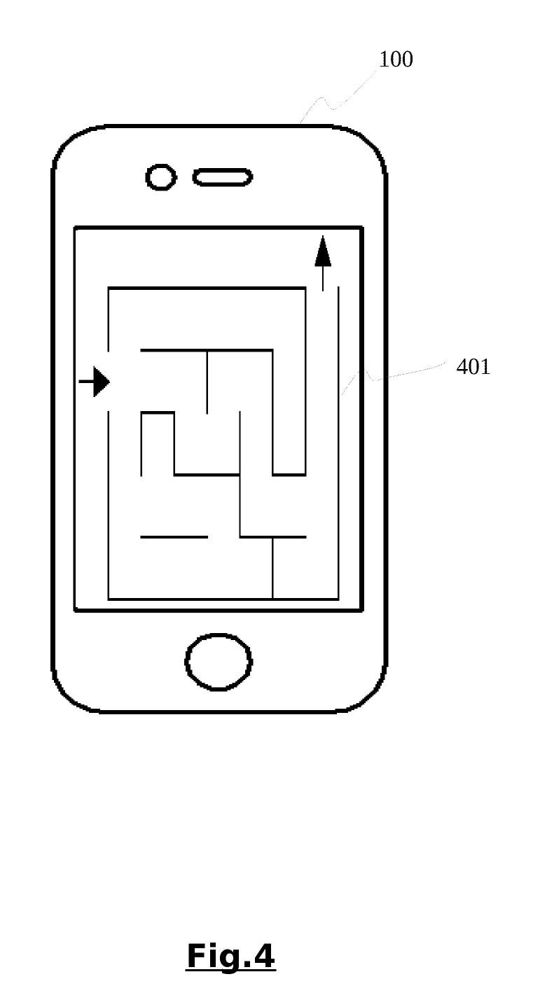

A further example of a possible test is shown in FIG. 4. Here a simple maze 401 is presented, which the user must navigate in order unlock a locked phone or tablet.

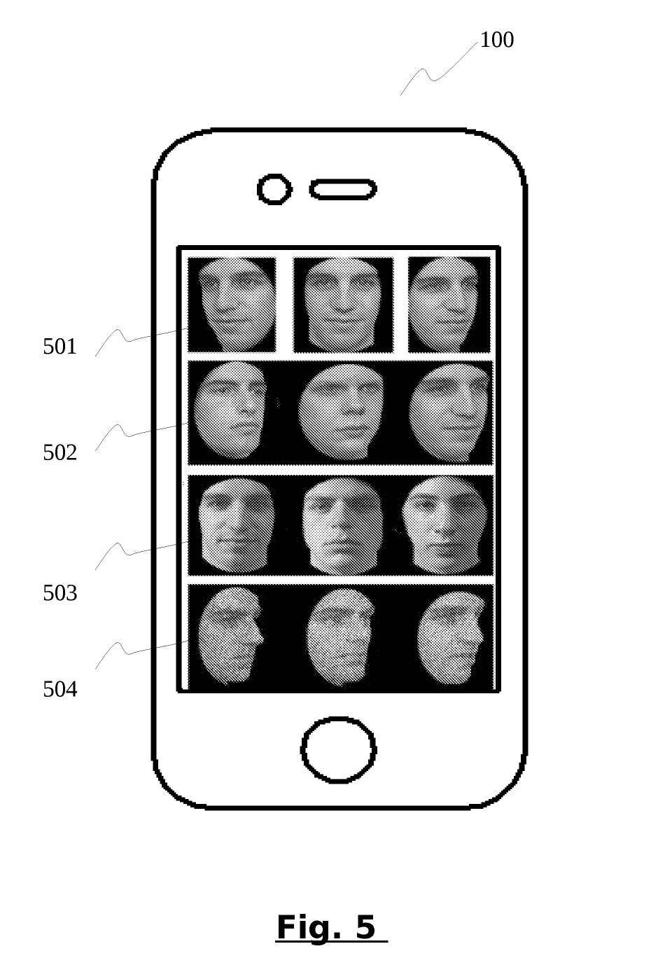

A still further example is shown in FIG. 5, wherein a set of faces is shown for a face matching test. The top row 501 shows a face to match to, from different angles. The second, third, and fourth rows 502,503,504 have sets of three where one face matches the target face in the top row. The user must choose the matching face in order to unlock the phone.

The results of the various tests are preferably sent to a networked server where they are stored in a database. The user and/or other entities may then analyze these results as they change over time. For instance, the effects of medication on sensory, motor, and cognitive function may be assessed by means of comparing these results before and after treatment. Such studies can assess responses from a number of individuals over a period of time to uncover environmental, temporal, geographical, and other effects.

The progress of neurodegenerative disease can be followed, and furthermore action can be taken as a result of the user performance; for example a user who is unable to perform a certain task may be flagged as a candidate for a neurological or other examination.

The progress of a patient's responses can be tracked not only over days and weeks but within a single day, as it is often the case that a user unlocks a phone several times a day at minimum. Updates on the scale of hours allows the system to respond to these changes for better stabilitzation, for example by means of recommending user actions (taking medications, getting professional assistance, doing exercise, or any of a host of possible recommended actions).

The perspicacious reader will note that since unlocking smartphone is a common-enough occurrence in any case, there will be little resistance to co-opting this common action for purposes of diagnostic testing. Furthermore compliance problems are avoided since the user is performing the various aactions required of their own volition.

The results of the diagnostic tests may be analyzed locally and/or remotely. Remote analysis may be performed automatically, manually, or by a combination of such methods. This analysis may be based on the current results and any set of previous results as well.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

1. A method of diagnostic testing consisring of the steps:
locking a computing device such that it cannot be used;
presenting a login challenge to a user of said computing device consisting of a test of said user's sensory, cognitive, and/or motor abilities;
recording the response to said login challenge;
allowing use of said computing device if said login challenge is passed;
whereby the use of a login challenge is leveraged to provide diagnostic information concerning said user.

2. The method of claim 1 using sensors selected from the group consisting of: a touch-sensitive surface of said computing device, accelerometer, magnetometer, inclinometer, gyro, GPS, camera, and microphone

3. The method of claim 1 wherein said challenge is selected from the group consisting of: a requirement to give a correct password or numerical code; a puzzle, mathematical question or textual riddle; identification of digits, objects, or faces; and combinations thereof.

4. The method of claim 1 wherein the results of said diagnostic tests are analyzed locally and/or remotely, by automatic, manually, or combinations of such means, based on the current results and any set of previous results.

5. The method of claim 4 wherein said results are used to provide diagnoses concerning said user, and to take actions selected from the group consisting of: contacting a concerned party; recommending action on the part of said user; placing such user on a watchlist; and changing the nature of said diagnostic test.
